(19) United States
(12) Patent Application Publication
(10) Pub. No.: US 2006/0029648 A1
(43) Pub. Date: Feb. 9, 2006

(54) Stimulant/desensitizer swabs

(76) Inventor: Garry Tsaur, Rowland Heights, CA (US)

Correspondence Address: JOE NIEH, 18760 E. AMAR ROAD #204, WALNUT, CA, 91789, US

(21) Appl. No.: 10/911,285

(22) Filed: Aug. 3, 2004

(51) Int. Cl. A61K 9/70; A61K 36/81; A61K 36/534

(52) U.S. Cl. 424/443; 424/747; 424/760

(57) ABSTRACT

A method and pharmaceutical composition for treating the sexual dysfunction of premature ejaculation is disclosed. The pharmaceutical composition consists of a solution of one or more stimulants and a desensitizer. The stimulant is selected from the group comprising spearmint solution, peppermint solution, cool mint solution, pepper solution, alcohol, rubbing alcohol, ether, or any solution that will stimulate the penis to cause an erection. The desensitizer is selected from the group comprising benzocaine and lidocaine. The pharmaceutical composition is enclosed within a cotton swab applicator and is applied directly to the penis with the cotton swab applicator.

### BACKGROUND—FIELD OF INVENTION

The present invention relates generally to a method and pharmaceutical composition for treating sexual dysfunction. More specifically, the present invention relates to treatment of premature ejaculation by local topical administration of selected pharmacologically active agents with a swab.

### BACKGROUND—DESCRIPTION OF RELATED ART

Premature ejaculation is a sexual dysfunction. This dysfunction can lead to an inability to enter or sustain relationships and can cause psychological damage to sufferers. Premature ejaculation can also impair reproductive success. It has been estimated that premature ejaculation affects up to 75 percent of the population. Premature ejaculation is also a considerable factor in sexual as well as marital discord. It is estimated that this factor is present in at least 20 percent of clinical cases. However, there has not been an effective and inexpensive treatment that can be administered by anyone without specialized knowledge or training.

Previous methods of treating premature ejaculation include psychological therapies, topical anesthetics, and the use of devices such as prosthetics. All these previous methods have significant drawbacks. Psychotherapy requires sexual training for a long period of time which involves discussions and cooperation with a physician and the patient and his partner. However, since psychotherapy necessitates a long period of time for the physician, patient, and his partner to work together in order to be effective the success rate of psychotherapy is low. Psychotherapies benefit only some of the patients and require specialized therapists who may not be available to all patients, particularly in remote areas. Furthermore, psychotherapies cannot alleviate premature ejaculation resulting from non-psychological causes.

Topical anesthetics are currently more widely used since time restrictions are not as great and it is more convenient to use. Anesthetic agents decrease sensitivity of tissues, thereby diminishing sexual pleasure. Also, topical anesthetics can be transferred to sexual partners and thereby decrease their sensitivity and pleasure as well.

Devices can be awkward, inconvenient, and embarrassing to use. Devices are highly conspicuous, and reveal the very condition which the suffering partner may prefer to conceal. Furthermore, devices usually require some practiced skill to use properly and effectively. Additionally, devices can cause irritation to one or both partners.

Thus there is a need for a method of treating premature ejaculation that requires no specialized psychological therapy, can be used conveniently and without embarrassment, and does not involve the problems associated with prior therapeutic methods.

### BRIEF SUMMARY OF THE INVENTION

The present invention is a method and pharmaceutical composition for treating the sexual dysfunction of premature ejaculation. The pharmaceutical composition consists of a solution of one or more stimulants and a desensitizer. The stimulant is selected from the group comprising spearmint solution, peppermint solution, cool mint solution, pepper solution, alcohol, rubbing alcohol, ether, or any solution that will stimulate the penis to cause an erection. The desensitizer is selected from the group comprising benzocaine and lidocaine. The pharmaceutical composition is enclosed within a cotton swab applicator and is applied directly to the penis with the cotton swab applicator. The stimulant will quickly stimulate the penis resulting in a fast erection while the desensitizer, which takes more time to affect the nerves, will desensitize the penis after erection to delay or prevent premature ejaculation.

### DETAILED DESCRIPTION OF THE PREFERRED EMBODIMENT

The following description and figures are meant to be illustrative only and not limiting. Other embodiments of this invention will be apparent to those of ordinary skill in the art in view of this description.

The present invention is a method and pharmaceutical composition for treating the sexual dysfunction of premature ejaculation. The pharmaceutical composition is a solution of one or more stimulants and a desensitizer. The stimulant is selected from the group comprising spearmint solution, peppermint solution, cool mint solution, pepper solution, alcohol, rubbing alcohol, ether, or any solution that will stimulate the penis to cause an erection. The desensitizer is selected from the group comprising benzocaine and lidocaine. In the preferred embodiment, the desensitizer consists of 3 to 7.5 percent benzocaine in a water-soluble base.

The pharmaceutical composition is enclosed within a cotton swab applicator and is applied directly to the penis with the cotton swab applicator. U.S. Pat. No. 5,702,035, issued to Chien-Hua Tsao, discloses the preferred embodiment of the cotton swab applicator. In the preferred embodiment, the cotton swab applicator comprises of an elongated tubular housing enclosing the pharmaceutical composition with an applicator end wrapped by an absorbent material and a sealed end. A viscous substance such as silicone is disposed near the applicator end to seal the pharmaceutical composition within the elongated tubular housing. Upon breaking the sealed end, atmospheric pressure will cause the pharmaceutical composition to flow out of the elongated tubular housing through the applicator end into the absorbent material for application. After direct topical application of the pharmaceutical composition to the penis, the stimulant will quickly stimulate the penis resulting in a fast erection while the desensitizer, which takes more time to affect the nerves, will desensitize the penis after erection to delay or prevent premature ejaculation.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A stimulant/desensitizer swab comprising a pharmaceutical composition enclosed within a cotton swab applicator wherein said pharmaceutical composition consists of one or more stimulants and a desensitizer.

2. A stimulant/desensitizer swab as in claim 1, wherein said cotton swab applicator comprises of an elongated tubular housing enclosing said pharmaceutical composition with an applicator end wrapped by an absorbent material and a sealed end wherein a viscous substance is disposed near said applicator end to seal said pharmaceutical composition within said elongated tubular housing whereby upon breaking said sealed end, atmospheric pressure will cause said pharmaceutical composition to flow out of said elongated tubular housing through said applicator end into said absorbent material for application.

3. A stimulant/desensitizer swab as in claim 1, wherein said stimulant is selected from the group comprising spearmint solution, peppermint solution, cool mint solution, pepper solution, alcohol, rubbing alcohol, ether, or any solution that will stimulate the penis to cause an erection.

4. A stimulant/desensitizer swab as in claim 1, wherein said desensitizer is selected from the group comprising benzocaine and lidocaine.

5. A stimulant/desensitizer swab as in claim 4, wherein said desensitizer consists of 3 to 7.5 percent benzocaine in a water-soluble base.

6. A pharmaceutical composition for treating the sexual dysfunction of premature ejaculation consisting one or more stimulant and a desensitizer.

7. A pharmaceutical composition for treating the sexual dysfunction of premature ejaculation as in claim 6, wherein said stimulant is selected from the group comprising spearmint solution, peppermint solution, cool mint solution, pepper solution, alcohol, rubbing alcohol, ether, or any solution that will stimulate the penis to cause an erection.

8. A pharmaceutical composition for treating the sexual dysfunction of premature ejaculation as in claim 6, wherein said desensitizer is selected from the group comprising benzocaine and lidocaine.

9. A pharmaceutical composition for treating the sexual dysfunction of premature ejaculation as in claim 8, wherein said desensitizer consists of 3 to 7.5 percent benzocaine in a water-soluble base.
